(19) United States
(12) Patent Application Publication
(10) Pub. No.: US 2009/0105646 A1
(43) Pub. Date: Apr. 23, 2009

(54) Multi-Frequency Communication System For A Drug Infusion Device

(75) Inventors: Charles Hendrixson, West Chester, PA (US); Bahram Sharifi, Schwenksville, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Animas Corporation, West Chester, PA (US)

(21) Appl. No.: 11/874,599

(22) Filed: Oct. 18, 2007

(51) Int. Cl. A61M 5/20

(52) U.S. Cl. 604/135

(57) ABSTRACT

Disclosed is a medical infusion device, such as an externally worn insulin pump, capable of being in remote communication with a controller or data acquisition unit such as a blood glucose meter. The disclosed medical infusion device includes a dual frequency antenna to facilitate communication with the remote device and the antenna is mounted using a spring-design that inhibits transmission of vibration to the antenna.

### CROSS REFERENCE TO RELATED APPLICATIONS

This application is related to copending, commonly assigned U.S. patent application Ser. No. 11/871,183 (not yet assigned), filed on Oct. 12, 2007 in the names of Charles Hendrixson and Bahram Sharifi.

### FIELD OF THE INVENTION

The present invention relates, in general, to drug delivery systems and, more particularly, to a communications system for a drug delivery device that may be remotely controlled. The present invention also relates to methods of assembling such a drug delivery device in a manner that improves reliability and reduces mechanical vibrations in the device.

### BACKGROUND OF THE INVENTION

Diabetes mellitus is a chronic metabolic disorder caused by an inability of the pancreas to produce sufficient amounts of the hormone insulin so that the metabolism is unable to provide for the proper absorption of sugar and starch. This failure leads to hyperglycemia, i.e. the presence of an excessive amount of glucose within the blood plasma. Persistent hyperglycemia causes a variety of serious symptoms and life threatening long term complications such as dehydration, ketoacidosis, diabetic coma, cardiovascular diseases, chronic renal failure, retinal damage and nerve damages with the risk of amputation of extremities. Because healing is not yet possible, a permanent therapy is necessary which provides constant glycemic control in order to always maintain the level of blood glucose within normal limits. Such glycemic control is achieved by regularly supplying external insulin to the body of the patient to thereby reduce the elevated levels of blood glucose.

External insulin was commonly administered by means of multiple, daily injections of a mixture of rapid and intermediate acting insulin via a hypodermic syringe. While this treatment does not require the frequent estimation of blood glucose, it has been found that the degree of glycemic control achievable in this way is suboptimal because the delivery is unlike physiological insulin production, according to which insulin enters the bloodstream at a lower rate and over a more extended period of time. Improved glycemic control may be achieved by the so-called intensive insulin therapy which is based on multiple daily injections, including one or two injections per day of long acting insulin for providing basal insulin and additional injections of rapidly acting insulin before each meal in an amount proportional to the size of the meal. Although traditional syringes have at least partly been replaced by insulin pens, the frequent injections are nevertheless very inconvenient for the patient, particularly those who are incapable of reliably self-administering injections.

Substantial improvements in diabetes therapy have been achieved by the development of the insulin infusion pump, relieving the patient of the need syringes or insulin pens and the administration of multiple, daily injections. The insulin pump allows for the delivery of insulin in a manner that bears greater similarity to the naturally occurring physiological processes and can be controlled to follow standard or individually modified protocols to give the patient better glycemic control.

Infusion pumps can be constructed as an implantable device for subcutaneous arrangement or can be constructed as an external device with an infusion set for subcutaneous infusion to the patient via the transcutaneous insertion of a catheter or cannula. External infusion pumps are mounted on clothing, hidden beneath or inside clothing, or mounted on the body and are generally controlled via a user interface built-in to the device.

Regardless of the type of infusion pump, blood glucose monitoring is required to achieve acceptable glycemic control. For example, delivery of suitable amounts of insulin by the insulin pump requires that the patient frequently determines his or her blood glucose level and manually input this value into a user interface for the external pumps, which then calculates a suitable modification to the default or currently in-use insulin delivery protocol, i.e. dosage and timing, and subsequently communicates with the insulin pump to adjust its operation accordingly. The determination of blood glucose concentration is typically performed by means of a measuring device such as a hand-held electronic meter which receives blood samples via enzyme-based test strips and calculates the blood glucose value based on the enzymatic reaction.

Since the blood glucose meter is an important part of an effective glycemic control treatment program, integrating the measuring aspects of the meter into an external pump or the remote of a pump is desirable. Integration eliminates the need for the patient to carry a separate meter device, it offers added convenience and safety advantages by eliminating the manual input of the glucose readings, and may reduce instances of incorrect drug dosaging resulting inaccurate data entry.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an illustrative schematic view of elements of a drug delivery system according to an exemplary embodiment of the invention.

FIG. 2 is a block diagram of a drug delivery system according to an exemplary embodiment of the invention.

FIG. 3 is a perspective view of a drug delivery device according to an exemplary embodiment of the invention.

FIG. 4 is a perspective, cross-sectional view of the drug delivery device shown in FIG. 3 with the drug reservoir cap, bolus button, battery cap, battery and vibrator removed.

FIG. 5 is a perspective view of a housing for a drug delivery device according to an exemplary embodiment with the drug reservoir cap, bolus button, battery cap and navigational buttons removed.

FIG. 6 is a perspective view of another housing for a drug delivery device with the display cover removed according to an exemplary embodiment.

FIG. 7 is a perspective view of a radio frequency module according to an exemplary embodiment of the invention.

FIGS. 8A and 8B are top and bottom views, respectively, of an antenna according to an exemplary embodiment of the invention.

FIGS. 9A, 9B and 9C are various views of spring connector configurations on the circuit board of the radio frequency module according to an exemplary embodiment of the invention.

FIG. 10 is a simplified schematic view of the drug delivery device shown in FIG. 3 according to an exemplary embodiment of the invention.

### DETAILED DESCRIPTION OF THE INVENTION

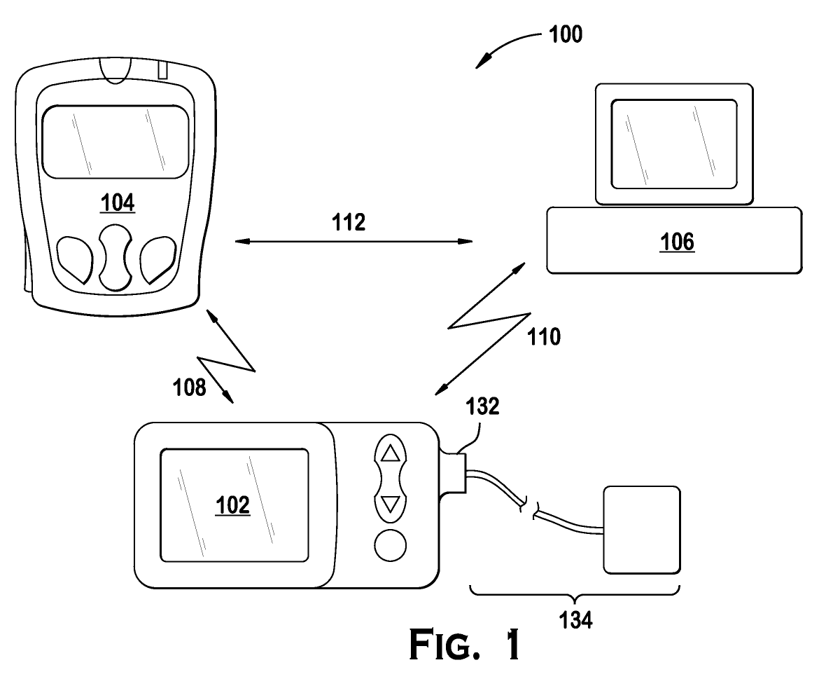

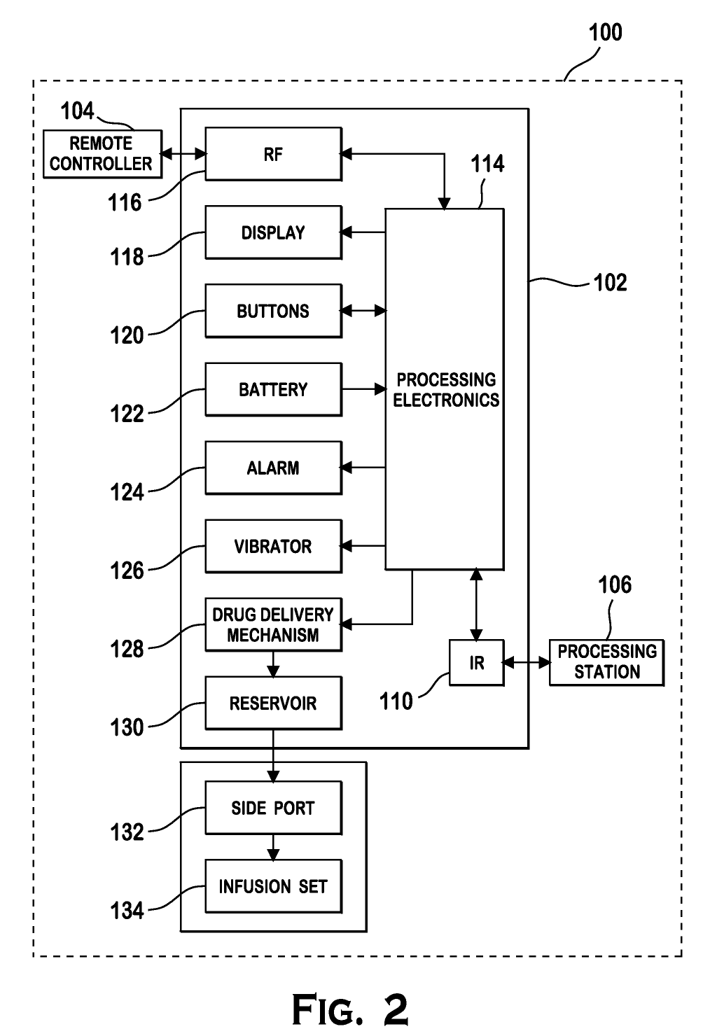

FIGS. 1 and 2 illustrate a drug delivery system 100 according to an exemplary embodiment. Drug delivery system 100 includes a drug delivery device 102, a remote controller 104 and an optional processing station 106. Drug delivery device 102 is configured to transmit and receive data to and from remote controller 104 by, for example, radio frequency communication 108. Drug delivery device 102 may also function as a stand-alone device with its own built in controller. In one embodiment, drug delivery device 102 is an insulin infusion device and remote controller 104 is a hand-held blood glucose metering system. In such an embodiment, data transmitted from drug delivery device 102 to remote controller 104 may include insulin delivery data. Data transmitted from remote controller 104 to drug delivery device 102 may include glucose test results and a food database to aid in calculating the amount of insulin to be delivered by drug delivery device 102. In another embodiment (not shown), remote controller 104 is a continuous metering system for detecting glucose in blood or interstitial fluid.

Drug delivery device 102 may also be configured for bi-directional wireless communication with processing station through, for example, an infrared signal 110. Remote controller 104 and processing station 106 may be configured for bi-directional wired communication through, for example, a universal serial bus (USB) cable 112. Processing station 106 may be used, for example, to download upgraded software to drug delivery device 102 and to process information from drug delivery device 102. Examples of processing station 106 may include, but are not limited to, a personal or networked computer, a personal digital assistant or a mobile telephone.

Referring to FIG. 2, drug delivery device 102 includes processing electronics 114 including a central processing unit and memory elements for storing control programs and operation data, a radio frequency module 116 for sending and receiving communication signals (i.e., messages) to/from remote controller 104, a display 118 for providing operational information to the user, a plurality of navigational buttons 120 for the user to input information, a battery 122 for providing power to the system, an alarm 124 for providing feedback to the user, a vibrator 126 for providing feedback to the user, a drug delivery mechanism 128 (e.g. an insulin pump and drive mechanism) for forcing a drug from a drug reservoir 130 (e.g., an insulin cartridge) through a side port 132 connected to an infusion set 134 and into the body of the user.

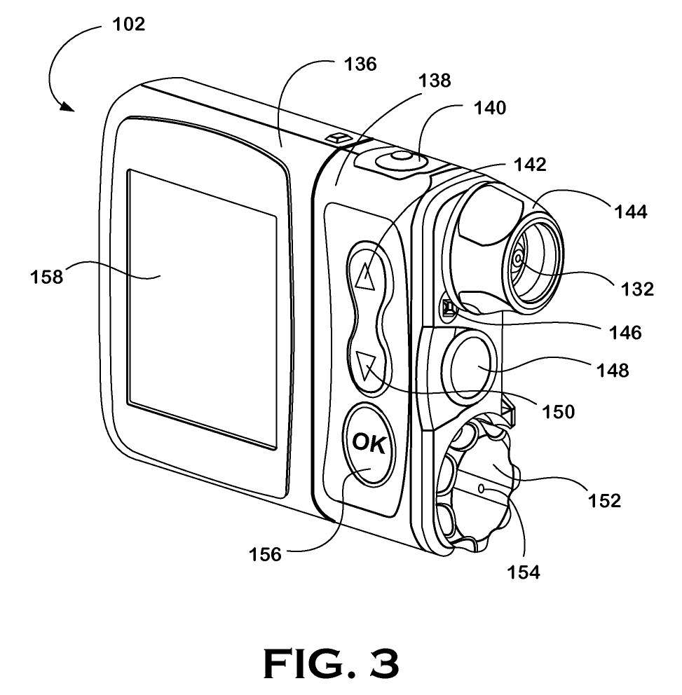

As illustrated in FIG. 3, drug delivery device 102 further includes a first housing 136, a second housing 138, a backlight button 140, an up button 142, a drug reservoir cap 144, a first primary vent 146, a bolus button 148, a down button 150, a battery cap 152 with a second primary vent 154, an OK button 156 and a display cover 158. First housing 136 and second housing 138 are typically formed from a durable plastic material.

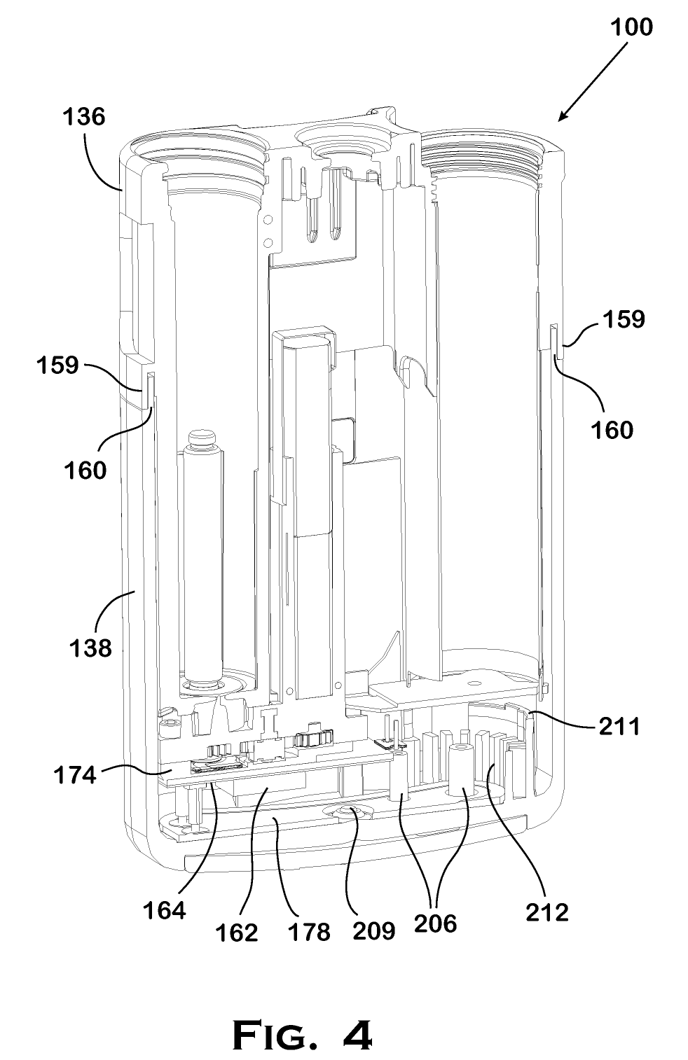

Referring to FIGS. 4, 5 and 6, first housing 136 is nested at least partially within second housing 138 and includes a grooved portion 159 that receives a tongue portion 160 of second housing 138. First housing 136 houses drug reservoir 130, drug delivery mechanism 128, processing electronics 114, battery 122, and a transceiver 162 mounted on a first surface 163 of a circuit board 164. Drug reservoir 130, drug delivery mechanism 128 with the electronics and battery 122 are each encased in sealed compartments in first housing 136. In one embodiment, a drug delivery mechanism/electronics compartment 166 of drug delivery device 102 is located between a drug reservoir compartment 168 and a battery compartment 170.

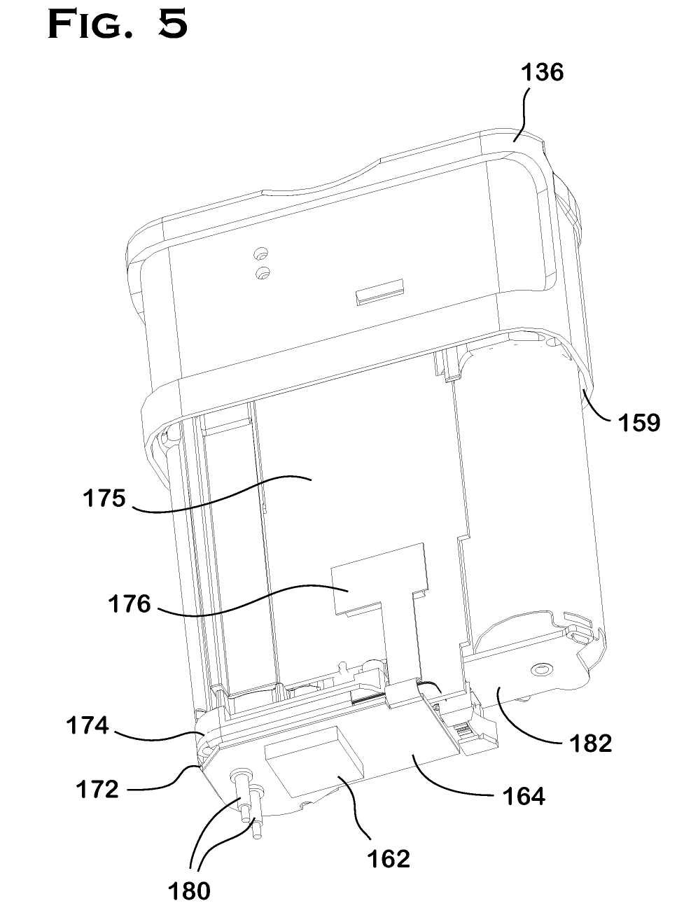

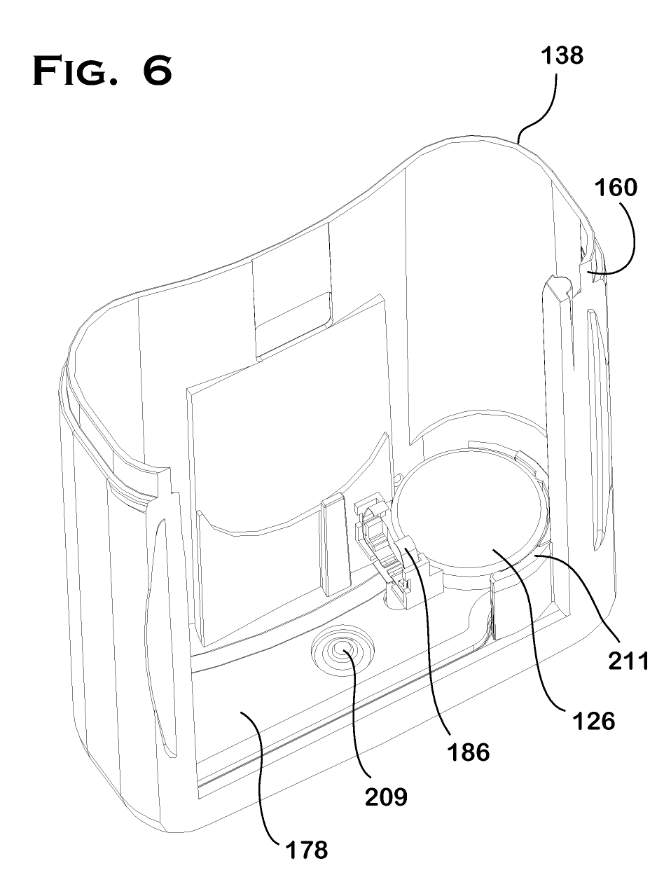

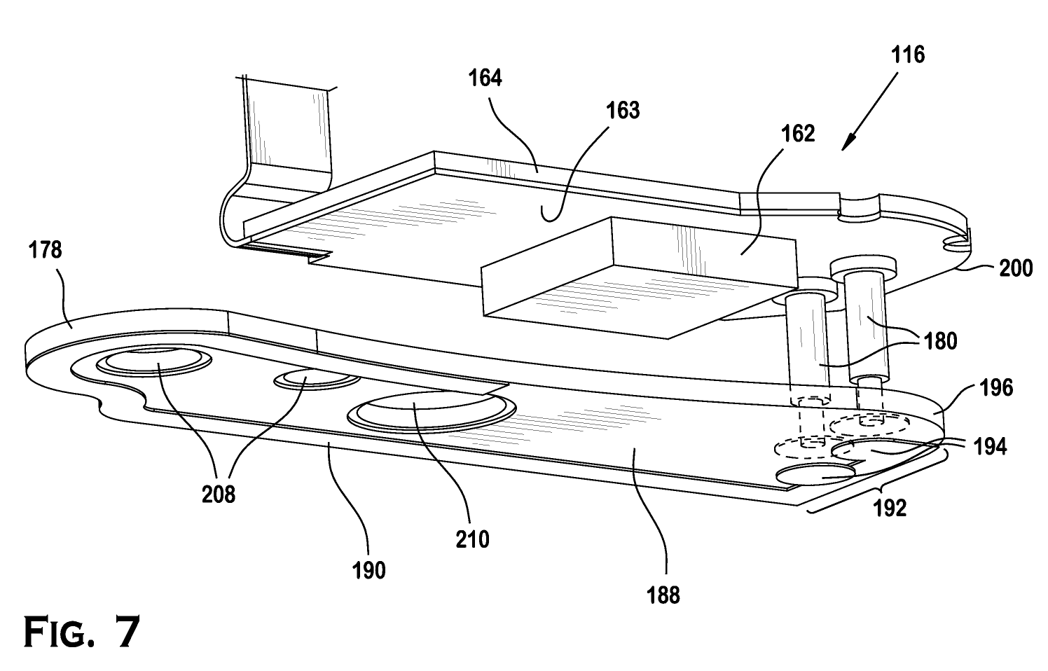

Located on a distal end 172 of first housing 136, circuit board 164 is connected to a gear plate 174 in drug delivery mechanism 128 and is operatively connected to the main circuit board of drug delivery device 102 through a board connector 176 (see FIGS. 4 and 5). Transceiver 162 is also operatively connected to an antenna 178 in second housing 138 by at least one spring connector 180 (e.g., a pogo pin). At least one spring connector 180 allows for ease of assembly of drug dispensing device. Together, transceiver 162 mounted on circuit board 164 and antenna 178 form radio frequency module 116 (see FIG. 7). Second housing 138 also includes vibrator 126 operatively connected to battery 122 by, for example, a spring clip 186 (see FIG. 6).

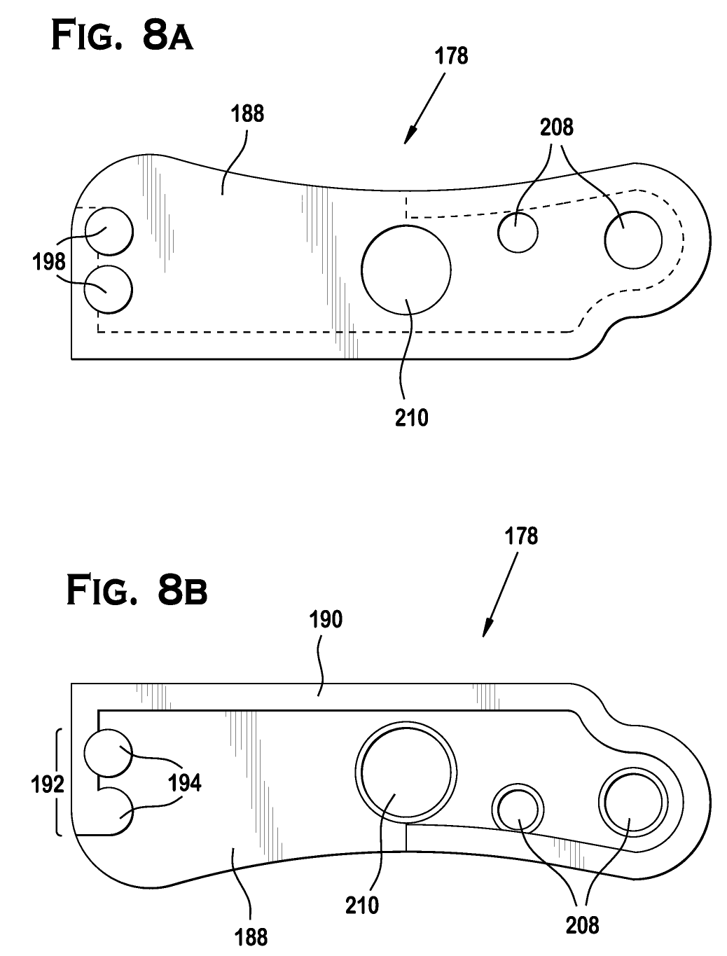

Referring to FIGS. 7, 8A and 8B, antenna 178 includes a substrate 188, a conductive trace 190 (i.e., the resonating portion), and a signal feed region 192. Trace 190 is electrically connected to a first conductive pad 194 in signal feed region 192 at or adjacent to a first end 196 of substrate 188. Substrate 188 provides a support for trace 190 and is manufactured from a dielectric material or a flexible material. For example, a small fiberglass-based printed circuit board may be used. Other examples of materials that may be used for substrate 188 include, but are not limited to, FR4 plastic, phenolic material and fiberglass reinforced Teflon. The use of a thin substrate 188 provides the advantage of being deformable and easily mounted in place.

Trace 190 is formed from a conductive material such as, for example copper, brass, aluminum, silver or gold. Trace 190 may be deposited onto substrate 188 using a technique known to those skilled in the art such as, but not limited to, photo-etching of a conductive material on a dielectric or insulated substrate, plating of a conductive material on a substrate, or adhering a conductive material, such as a thin plate of metal, on a substrate with adhesive.

The length of trace 190 primarily determines the resonant frequency of antenna 178. Trace 190 is sized appropriately for a particular operating frequency. Traces 190 used to form the antenna 178 are deposited to provide a conductive element that is approximately ¼ an effective wavelength (λ) for the frequency of interest. Those skilled in the art will readily recognize the benefits of making the length slightly greater or less than λ/4, for purposes of matching the impedance to corresponding transmit or receive circuitry. In addition, connecting elements such as exposed cables, wires, or the spring connector 180 contribute to the overall length of antenna 178, and are taken into account when choosing the dimensions of trace 190.

Where antenna 178 is used with a wireless device capable of communicating at more than one frequency, the length of trace 190 is based on the relationship of the frequencies. That is, multiple frequencies can be accommodated provided they are related by fractions of a wavelength. For example, the λ/4 length for one frequency corresponds to 3λ/4 or λ/2 for the second frequency.

The width of trace 190 is less than a wavelength in the dielectric substrate material so that higher-order modes will not be excited. In the embodiment shown in FIGS. 7 and 8B, width of trace 190 is between about 0.5 to 2.0 millimeters, typically about 1.5 millimeters. In the subject invention, the length and width of trace 190 is sized so that antenna 178 is capable of receiving and transmitting signals having a frequency range between about 850 MHz and about 950 MHz. In one embodiment, antenna 178 may transmit and receive signals in the frequency range between about 869.70 MHz and about 870 MHz. In another embodiment, antenna 178 may transmit and receive signals in the frequency range between about 902 MHz and about 928 MHz. In another illustrative embodiment, the antenna 178 may transmit and/or receive at a first and second frequency range at the same time. Illustratively, the first frequency range may comprise wavelengths of between about 869.70 MHz and about 870 MHz and the second frequency range may comprise wavelengths of between about 902 MHz and about 928 MHz

The thickness of trace 190 is usually on the order of a small fraction of the wavelength, in order to minimize or prevent transverse currents or modes, and to maintain a minimal antenna 178 size (i.e., thickness). The selected value is based on the bandwidth over which antenna 178 must operate.

The total length of trace 190 is approximately λ/4, but it should be noted that trace 190 may be folded, bent, or otherwise redirected, to extend back along the direction it came so that the overall antenna 178 size is reduced. As shown in FIG. 8B, trace 190 extends along the length and edge of substrate 188 such that it is redirected back toward first conductive pad 194. This allows antenna 178 to have a shorter overall length. The thin conductor dimensions combined with a relatively thin support substrate 188 and λ/4 total length allows a reduction in the overall size of antenna 178 compared to conventional strip or patch antennas, making it more desirable for use in portable medical devices. In one embodiment, the length of antenna 178 is about 41 millimeters and the widest portion of antenna 178 is about 13 millimeters.

As illustrated in FIGS. 7 and 8B, a first conductive pad 194 is positioned in signal feed region 192 and electrically coupled or connected to trace 190. Generally, first conductive pad 194 and trace 190 are formed from the same material, possibly as a single unified body or structure, using the same manufacturing technique, although this is not required. First conductive pad 194 simply needs to make good electrical contact with trace 190 for purposes of signal transfer without adversely impacting antenna impedance or performance.

In the antenna embodiment illustrated in FIGS. 7, 8A and 8B, which is a planar inverted-F antenna, trace 190 faces away from transceiver 162 such that substrate 188 is positioned between trace 190 and transceiver 162. In this situation, first conductive pad 194 is positioned on the side of substrate 188 that does not readily accept a signal directly from transceiver 162. Thus, as shown in FIG. 8A, a second conductive pad 198 may be used on the opposing side of substrate 188 and conductive vias (not shown) may be used to transfer signals through substrate 188.

The use of first conductive pad 194 and second conductive pad 198 allows antenna 178 to be installed and operated in a manner that provides for convenient electrical connection and signal transfer through the at least one spring connector 180 (e.g., pogo pins). This simplifies construction and manufacture of drug delivery device 102 by eliminating the need for manual installation of specialized connectors, or having to manually insert antenna 178 within a contact structure. To assemble radio frequency module, first housing 136 and second housing 138 are simply snap-fitted together (e.g. tongue portion 160 of second housing 138 is fit into grooved portion 159 of first housing 136). To ensure a watertight fit, first housing 136 and second housing 138 may then be adhered together by adhesive. Having spring connectors also eliminates the need for a separate antenna housing that would be attached (e.g., glued) to drug delivery device 102 in an additional manufacturing step. Because a separately attached antenna housing is not needed, a possible source of water ingress is eliminated.

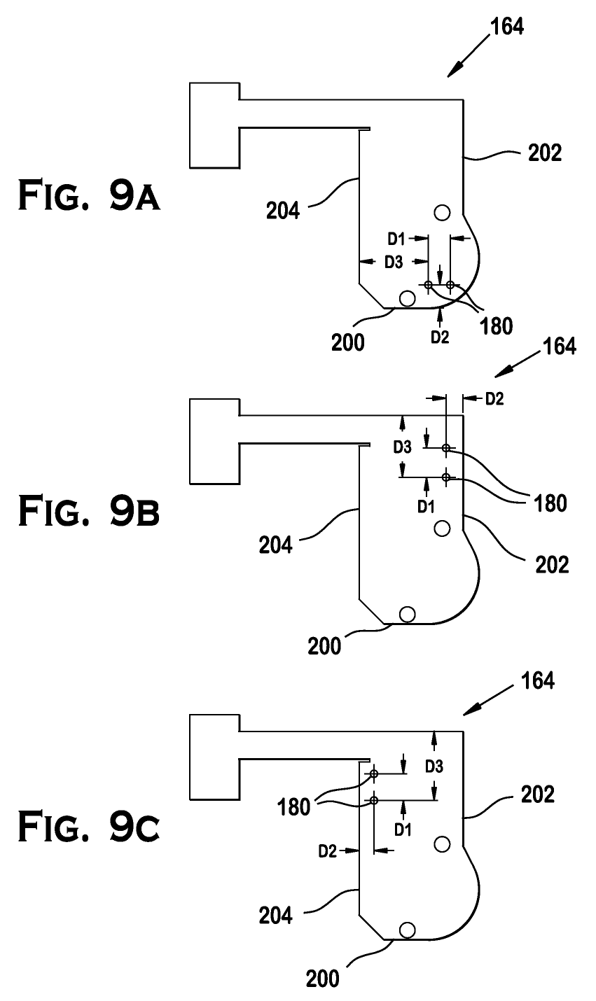

Antenna 178 is mounted in drug delivery device 102 adjacent to transceiver 162 and is placed substantially parallel to the ground plane provided by circuit board 164. Second conductive pad 198 is positioned adjacent to and electrically coupled to circuit board 164 using at least one spring connector 180. At least one spring connector 180 is mounted on circuit board 164 by, for example, soldering or conductive adhesives. As illustrated in FIGS. 7 and 9A, at least one spring connector 180 may be mounted near a first end 200 of circuit board 164. At least one spring connector 180 may also be mounted near a first edge 202 of circuit board 164 or near a second edge 204 of circuit board 164, depending on where antenna 178 is located in drug delivery device 102 (see FIGS. 9B and 9C). Generally, a distance D1 between two spring connectors 180 is between about 2.5 millimeters and about 4 millimeters. A distance D2 from two spring connectors to an edge of circuit board 164 parallel to a line through two spring connectors 180 is between about 1.5 millimeters and about 5 millimeters. A distance D3 from a spring connector to an edge of circuit board 164 perpendicular to a line through two spring connectors 180 is between about 5 millimeters and about 13 millimeters.

At least one spring connector 180 is electrically connected on one end to appropriate conductors or conductive vias to transfer signals to and from circuit board 164. The other end of at least one spring connector 180 is generally free floating and extends from circuit board 164 toward contact pad of antenna 178. At least one spring connector 180 may be formed from a metallic material such as copper or brass.

As illustrated in FIGS. 4 and 6, antenna 178 is sized to occupy the entire inner surface of a distal end of second housing 138 to maximize the signal transmitted and received. Antenna 178 may be located on any inner surface of drug delivery device 102 as long as the signal transmitted and received is not blocked. In one embodiment, the location and size are such that the signal range of antenna 178 is about 3 meters when drug delivery device 102 is not held in the user's hand and is about 1 meter when drug delivery device 102 is held in the user's hand. The thickness of antenna 178 is such that length of drug delivery device 102 is kept to a minimum. In one embodiment, the thickness of antenna 178 is between about 0.6 and about 0.8 millimeters, typically about 0.76 millimeters, and the length of drug delivery device 102 is between about 7 and 9 centimeters, typically about 8 centimeters.

At least one protrusion 206 on an inner surface of second housing 138 protrudes through at least one hole 208 in substrate 188 of antenna 178. At least one protrusion 206 positions vibrator 126 above antenna 178 such that vibration generated by vibrator 126 does not interfere significantly with signals transmitted and received by antenna 178. In one embodiment, antenna 178 is located between about 4 millimeters and about 7 millimeters (typically about 5 millimeters) from the bottom edge of vibrator 126. At least one protrusion 206 serves as a conduit for vibration to be transferred to second housing 138. At least one protrusion 206 also serves as a simple mounting mechanism for positioning antenna 178 within distal end of second housing 138 of drug delivery device 102. An optional nodule 209 on the inner surface of the distal end of second housing 138 may also aid in positioning antenna within drug delivery device 102. Nodule 209 may protrude through a substrate opening 210 in antenna 178. A half cradle 211 formed from a plurality of ribs 212 in second housing 138 also supports vibrator 126 and transfers vibration radially from vibrator 126 to second housing 138 without interfering significantly with signals transmitted and received by antenna 178.

First housing 136 also includes a plurality of vents with water impermeable membranes to protect the internal components of drug delivery device 102 from water damage during such user activities as, for example, swimming. The water impermeable membranes are also air permeable to ensure rapid pressure equilibration between the interior of drug delivery device 102 and atmosphere that could cause unexpected and undesirable delivery of a drug to the user. A rapid pressure change may occur, for example, when a user flies in an airplane.

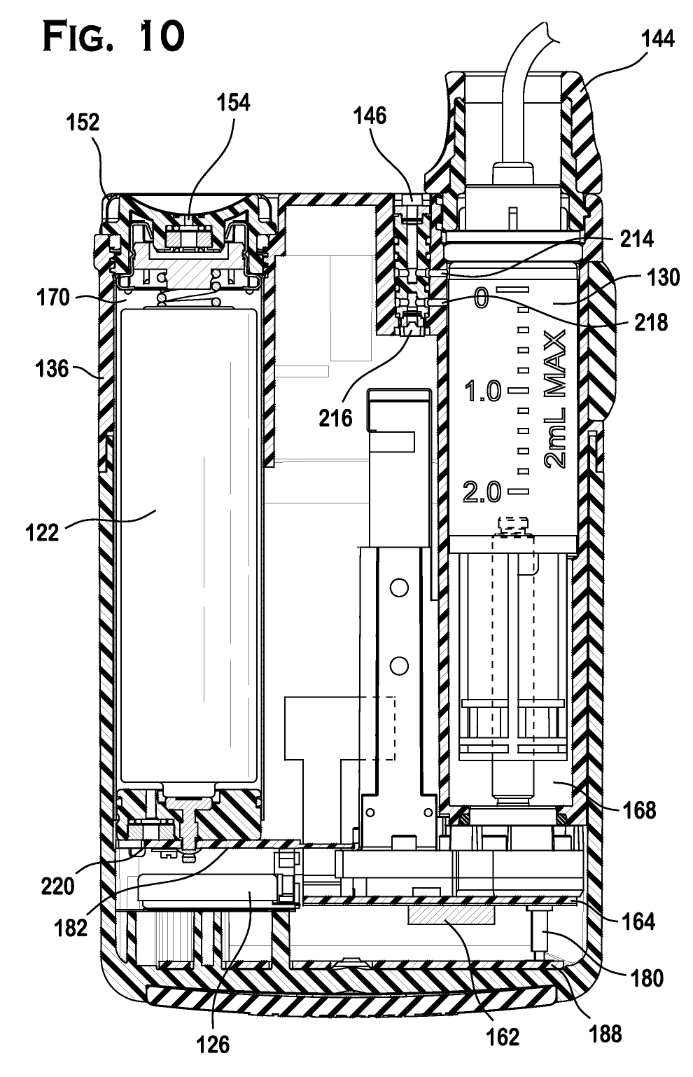

Referring to FIG. 10, first primary vent 146 is located in first housing 136 near drug reservoir cap 144 and vents the drug reservoir compartment to atmosphere through a first opening 214 into drug reservoir compartment 168. First primary vent 146 vents drug reservoir compartment 168 to atmosphere to ensure that there is no differential pressure between drug reservoir compartment 168 and atmosphere, which could result in unwanted dispensing of the drug from drug reservoir 130. Second primary vent 154 is located in battery cap 152 and vents battery compartment 170 to atmosphere. Second primary vent 154 prevents uncontrolled pressure build up of gas in battery compartment 170. For example, hydrogen gas resulting from a chemical reaction in battery 122 may build up in battery compartment 170.

A first secondary vent 216 is located between drug reservoir compartment 168 and drug delivery mechanism/electronics compartment 166 to equalize pressure inside drug delivery device 102. First secondary vent 216 vents the inside of drug delivery device 102 through a second opening 218 into drug reservoir compartment 168 (see FIG. 10). A second secondary vent 220 is located in distal end of battery compartment 170, i.e., near the positive terminal. Second secondary vent 220 provides a vent between battery compartment 170 and the drug delivery mechanism/electronics compartment 166 to equalize pressure inside drug delivery device 102.

Redundancy created by the presence of first primary vent 146, second primary vent 154, first secondary vent 216 and second secondary vent 220 ensures venting and pressure equilibration of all drug delivery device compartments (i.e., drug delivery mechanism/electronics 166, drug reservoir compartment 168 and battery compartment 170), even during abnormal situations such as occlusion of any of the primary or secondary vents.

The water impermeable membrane (e.g, a hydrophobic membrane) included in all the primary and secondary vents is selected such that the water entry pressure exceeds a fluid pressure at a selected depth, i.e., the depth to which the membrane can reasonably expect to be exposed upon immersion in water. For example, in the case in which a test pressure of 5.2 pounds per square inch (psi) is requested (i.e., water pressure at a depth of 12 feet below the surface), a selected water entry pressure of approximately 10 to 15 psi provides an exemplary design margin. Exemplary membrane materials include, but are not limited to, Emflon® and Mupor® polytetrafluoroethylene (PTFE).

While embodiments of the present invention have been shown and described herein, it will be obvious to those skilled in the art that such embodiments are provided by way of example only. Numerous variations, changes, and substitutions will now occur to those skilled in the art without departing from the invention.

It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A drug delivery device comprising:
a housing; and
a radio frequency module disposed in the housing and configured for wireless data transmission, the radio frequency module comprising:
a dual-frequency antenna configured to operate in a first frequency range and a second frequency range; and
a transceiver mounted on a circuit board that is reversibly connected to the antenna by at least one spring connector.

2. The drug delivery device of claim 1, wherein the at least one spring connector is a pogo pin.

3. The drug delivery device of claim 1, wherein the antenna occupies the inner distal surface of the drug delivery device.

4. The drug delivery device of claim 1, wherein the first frequency range comprises wavelengths of between about 869.70 MHz and about 870 MHz and the second frequency range comprises wavelengths of between about 902 MHz and about 928 MHz.

5. The drug delivery device of claim 1, wherein the antenna is a planar inverted-F antenna.
